(19) United States
(12) Patent Application Publication
(10) Pub. No.: US 2017/0340427 A1
(43) Pub. Date: Nov. 30, 2017

(54) NERVE SCAFFOLD

(71) Applicant: DONGGUAN DIANFU PRODUCT DESIGN CO., LTD., Dongguan (CN)

(72) Inventor: Yangde LI, Dongguan (CN)

(21) Appl. No.: 15/636,690

(22) Filed: Jun. 29, 2017

Related U.S. Application Data
(63) Continuation-in-part of application No. PCT/CN2015/082154, filed on Jun. 24, 2015.

(30) Foreign Application Priority Data
Dec. 29, 2014 (CN) 201420851211.1

(51) Int. Cl. A61F 2/00; A61F 2/02; A61L 27/58; A61L 27/04; A61L 27/28; A61L 27/56

(52) U.S. Cl. CPC A61F 2/0077; A61L 27/28; A61L 27/56; A61L 27/58; A61L 27/04; A61F 2/02; A61F 2002/0086; A61F 2210/0004; A61F 2230/0069; A61F 2230/0082

(57) ABSTRACT

A nerve scaffold, including a membrane material and a degradable metal wire. The degradable metal wire is enclosed in the membrane material. The membrane material is biodegradable material, and includes three-dimensional pore structure. The degradable metal wire is horizontally and vertically distributed in the nerve scaffold.

### CROSS-REFERENCE TO RELATED APPLICATIONS

This application is a continuation-in-part of International Patent Application No. PCT/CN2015/082154 with an international filing date of Jun. 24, 2015, designating the United States, now pending, and further claims foreign priority benefits to Chinese Patent Application No. 201420851211.1 filed Dec. 29, 2014. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND OF THE INVENTION

### Field of the Invention

The invention relates to a solid nerve scaffold comprising a metal wire.

### Description of the Related Art

Peripheral nerve injury or rupture caused by trauma often leads to the decline or loss of sensation and motor function. Often, it becomes necessary to bridge a gap formed when reconstructing the damaged peripheral nerve.

Conventionally, autografts and artificial nerve scaffolds have been used for that purpose. Autografts are preferred but contribute to morbidity. Artificial nerve scaffolds, on the other hand, are made of non-degradable materials which may cause nerve compression and necessitate revisions.

In recent years, the development and research has mainly focused on biodegradable nerve scaffolds. These materials can replace portions of an injured nerve; however, they only provide a conduit for the growth of axons, but do not promote regeneration of nerves per se.

### SUMMARY OF THE INVENTION

In view of the above-described problems, it is one objective of the invention to provide a solid nerve scaffold comprising a degradable metal wire. The nerve scaffold has a three-dimensional structure that is favorable to nerve regeneration, can promote and guide nerve growth, appropriately matches the implantation environment in terms of mechanical properties, and is biodegradable.

To achieve the above objective, in accordance with one embodiment of the invention, there is provided a nerve scaffold, comprising a membrane material; and a degradable metal wire. The degradable metal wire is enclosed in the membrane material; the membrane material is biodegradable material, and comprises three-dimensional pore structure; and the degradable metal wire is horizontally and vertically distributed in the nerve scaffold.

In a class of this embodiment, the nerve scaffold is cylindrical, strip, or flaky in shape.

In a class of this embodiment, the nerve scaffold is between 4 and 30 mm in length.

In a class of this embodiment, the three-dimensional pore structure accounts for 10-65% of the nerve scaffold.

In a class of this embodiment, the degradable metal wire is magnesium, zinc, calcium, iron, or an alloy thereof.

In a class of this embodiment, the membrane material is biodegradable material selected from the group consisting of collagen, polylactic acid, chitosan, hydroxyapatite, or a composite thereof.

In a class of this embodiment, the membrane material is originated from an animal blood vessel, lymph vessel, nerve canal, intestine, or muscle, of which the cells are extracted chemically.

Advantages of the nerve scaffold according to embodiments of the invention are summarized as follows: the nerve scaffold has a three-dimensional structure favorable to nerve regeneration, can promote and guide the nerve growth, has good mechanical property, is biodegradable, and is significant for the neural restoration.

### BRIEF DESCRIPTION OF THE DRAWINGS

FIG. 1 is a schematic diagram of a solid nerve scaffold comprising a degradable metal wire in accordance with one embodiment of the invention; and

FIG. 2 is a schematic diagram of a solid nerve scaffold comprising a degradable metal wire in accordance with another embodiment of the invention.

### DETAILED DESCRIPTION OF THE EMBODIMENTS

For further illustrating the invention, experiments detailing a solid nerve scaffold comprising a degradable metal wire are described hereinbelow combined with the drawings. It should be noted that the following examples are intended to describe and not to limit the invention.

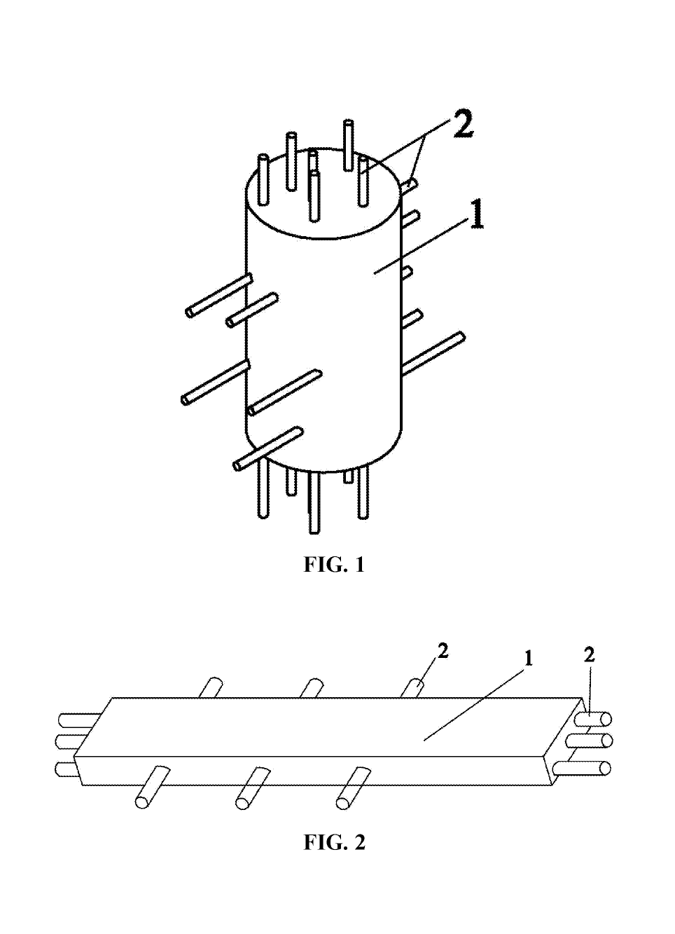

As shown in FIG. 1, a solid nerve scaffold comprising a degradable metal wire comprises a membrane material 1 and a degradable metal wire 2 which is enclosed in the membrane material 1. The degradable metal wire 2 is horizontally and vertically distributed in the nerve scaffold. The nerve scaffold is between 4 and 30 mm in length.

The nerve scaffold can be manufacture into desired shape as needed, in general, it is cylindrical, strip, or flaky in shape. FIG. 1 shows a cylindrical scaffold and FIG. 2 shows a flaky scaffold. It should be noted that, the shape of the nerve scaffold of the present disclosure is not limited to the above shape, and the shape of the nerve support is flexible and variable without departing from the basic conception of the invention.

The membrane material is biodegradable material, and comprises three-dimensional pore structure. The membrane material is biodegradable material selected from the group consisting of collagen, polylactic acid, chitosan, hydroxyapatite, or a composite thereof. The membrane material is originated from an animal blood vessel, lymph vessel, nerve canal, intestine, or muscle, of which the cells are extracted chemically.

The membrane material comprises three-dimensional pore structure. The three-dimensional pore structure accounts for 10-65% of the nerve scaffold, and provides space for the nerve growth.

The degradable metal wire 2 is magnesium, zinc, calcium, iron, or an alloy thereof, which can promote and guide the nerve growth.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A nerve scaffold, comprising:
a membrane material; and
a degradable metal wire;
wherein
the degradable metal wire is enclosed in the membrane material;
the membrane material is biodegradable material, and comprises three-dimensional pore structure; and
the degradable metal wire is horizontally and vertically distributed in the nerve scaffold.

2. The scaffold of claim 1, wherein the nerve scaffold is cylindrical, strip, or flaky in shape.

3. The scaffold of claim 1, wherein the nerve scaffold is between 4 and 30 mm in length.

4. The scaffold of claim 1, wherein the three-dimensional pore structure accounts for 10-65% of the nerve scaffold.
